(19) United States
(12) Patent Application Publication
(10) Pub. No.: US 2024/0173576 A1
(43) Pub. Date: May 30, 2024

(54) SKIN CARE DEVICE FOR USE IN BOTH ULTRASOUND DELIVERY AND DRUG APPLICATION

(71) Applicant: SKINGRAB CO., LTD., Seoul (KR)

(72) Inventor: Sugun LEE, Paju-si (KR)

(73) Assignee: SKINGRAB CO., LTD., Seoul (KR)

(21) Appl. No.: 18/429,910

(22) Filed: Feb. 1, 2024

Related U.S. Application Data
(63) Continuation of application No. PCT/KR2022/011569, filed on Aug. 4, 2022.

(30) Foreign Application Priority Data
Aug. 4, 2021 (KR) 10-2021-0102301
Jul. 27, 2022 (KR) 10-2022-0093198

(51) Int. Cl. A61N 7/02; A61N 7/00

(52) U.S. Cl. CPC A61N 7/02; A61N 2007/0034

(57) ABSTRACT

The present invention relates to a skin care device for use in both ultrasound delivery and drug application. The skin care device including at least: a cartridge that includes a head part contacting a target portion of the skin and a drug discharge part formed in the head part; an output unit that outputs, toward the target portion, electrical energy including ultrasound, laser, light, low-frequency microwave, medium-frequency microwave, or high-frequency microwave; a container loading unit that is detachably coupled to the head part; a drug solution container that stores a drug solution; and a needle unit that is provided in the container loading unit to allow the container loading unit to communicate with the drug discharging part, and breaks the lid of the drug solution container so that the drug solution stored in the drug solution container is applied to the target portion.

### CROSS-REFERENCE TO RELATED APPLICATIONS

The present application is a continuation of International Patent Application No. PCT/KR2022/011569, filed on Aug. 4, 2022, which is based upon and claims the benefit of priority to Korean Patent Application Nos. 10-2021-0102301 filed on Aug. 4, 2021 and 10-2022-0093198 filed on Jul. 27, 2022. The disclosures of the above-listed applications are hereby incorporated by reference herein in their entirety.

### BACKGROUND

Embodiments of the present disclosure described herein relate to a skin care device for both ultrasound delivery and drug application.

Skin care devices have been developed to remove wrinkles, restore skin elasticity, and remove sebum. This is because skin that is wrinkle-free, taut, thick, dense, and free of sagging makes a person look younger and contributes to an attractive appearance.

There are several types of skin care devices using a method of outputting a high-intensity focused ultrasound to a skin (HIFU type), a method of delivering a high frequency wave to the skin (RF type), a method of irradiating laser light to the skin (Optical type), and the like.

In this way, when electrical energy of the high-intensity focused ultrasound, the high frequency wave, and the laser light is delivered to the skin, damaged collagen and elastic fibers may be removed from the skin, new formation may be promoted, and skin pigmentation, acne marks, and wrinkles may be resolved.

Meanwhile, a drug is applied to the skin to relieve pain and treat microscopic wounds when the electrical energy is delivered to the skin.

However, a skin care device according to the related art has a problem in that the ultrasound cannot be delivered to the skin and the drug cannot be applied to the skin at the same time.

### SUMMARY

Embodiments of the present disclosure provide a skin care device for both ultrasound delivery and drug application, which may deliver electrical energy and apply a drug to a target part of a skin.

The aspects of the present disclosure are not limited to the aspects described above, and those skilled in the art will clearly understand other aspects not described from the following description.

According to an embodiment, a skin care device for both ultrasound delivery and drug application includes a cartridge including a head part in contact with a target part of a skin and a drug discharge portion formed in the head part, an output unit that is provided in the head part and outputs electrical energy including at least one of ultrasound, laser, light, a low-frequency microwave, a mid-frequency microwave, and a high-frequency microwave toward the target part, a container loading unit detachably coupled to the head part, a drug solution container which is loaded on the container loading unit and in which a drug solution is stored, and a needle part that is provided in the container loading unit to communicate with the container loading unit and the drug discharge portion and breaks a stopper of the drug solution container loaded on the container loading unit to apply the drug solution stored in the drug solution container to the target part.

Further, the skin care device may further include a container packing provided in the drug solution container to reciprocate, and a plunger that pushes the container packing in a direction approaching the needle part to discharge the drug solution stored in the drug solution container to the drug discharge portion.

Further, the skin care device may further include a handpiece detachably coupled to the cartridge and a driving unit that is provided in the handpiece and moves the plunger in a direction approaching or away from the needle part.

Further, the cartridge may include a drug flow portion which is recessed from the drug discharge portion in the head part in a vertical direction and through which the drug discharged from the drug discharge portion to the target part flows.

Further, the output unit may have an annular shape and may be disposed around the drug discharge portion.

The specific details of the present disclosure are included in the detailed description and the drawings.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a perspective view illustrating a state in which a skin care device for both ultrasound delivery and drug application according to an embodiment of the present disclosure is in contact with a skin;

FIG. 2 is a perspective view illustrating the skin care device for both ultrasound delivery and drug application according to the embodiment of the present disclosure;

FIG. 3 is a cross-sectional view illustrating the skin care device for both ultrasound delivery and drug application according to the embodiment of the present disclosure;

FIG. 4 is an exploded perspective view illustrating the skin care device for both ultrasound delivery and drug application according to the embodiment of the present disclosure;

FIGS. 5 to 6 are cross-sectional views illustrating a process of loading a drug solution container of a skin care device for both ultrasound delivery and drug application onto a container loading unit according to another embodiment of the present disclosure; and

FIG. 7 is a cross-sectional view illustrating the skin care device for both ultrasound delivery and drug application according to another embodiment of the present disclosure.

### DETAILED DESCRIPTION

Advantages and features of the present disclosure and a method of achieving the advantages and the features will become apparent with reference to embodiments described below in detail in conjunction with the accompanying drawings. However, the present disclosure is not limited to the embodiments described below but may be implemented in various forms, and the present embodiments merely make the disclosure of the present disclosure complete and are provided to completely inform the scope of the present disclosure to those skilled in the art to which the present disclosure belongs, and the present disclosure is merely defined by the scope of the appended claims.

Unless otherwise defined, all the terms (including technical and scientific terms) used herein may be used as meanings that may be commonly understood by those skilled in the art to which the present disclosure belongs. Further, terms defined in a commonly used dictionary are not interpreted ideally or excessively unless explicitly and specifically defined.

Hereinafter, embodiments of the present disclosure will be described in detail with reference to the accompanying drawings. In display of an end of the present disclosure, a tip may be an end adjacent to a target part of a skin, and a rear end may be an end that is not adjacent to the target part of the skin.

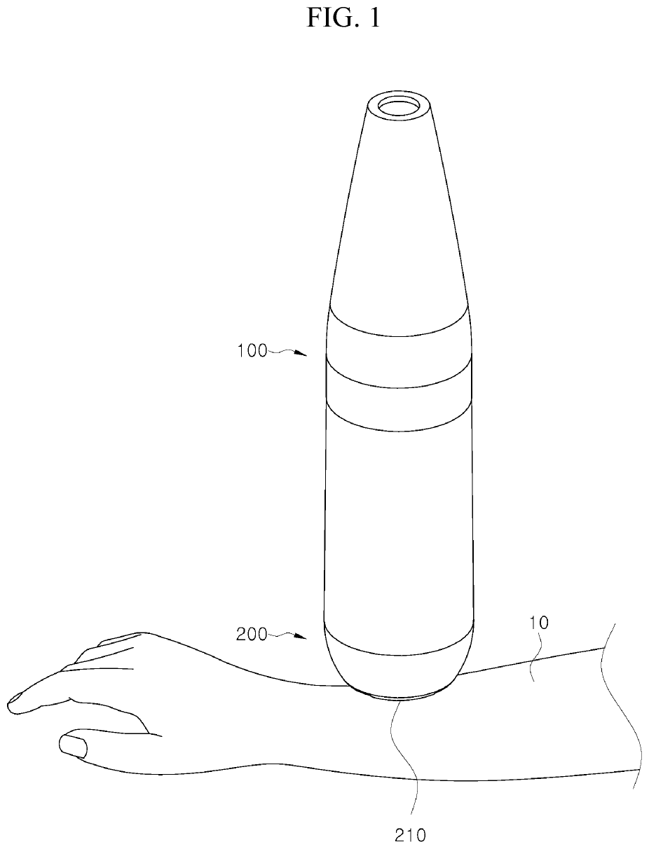

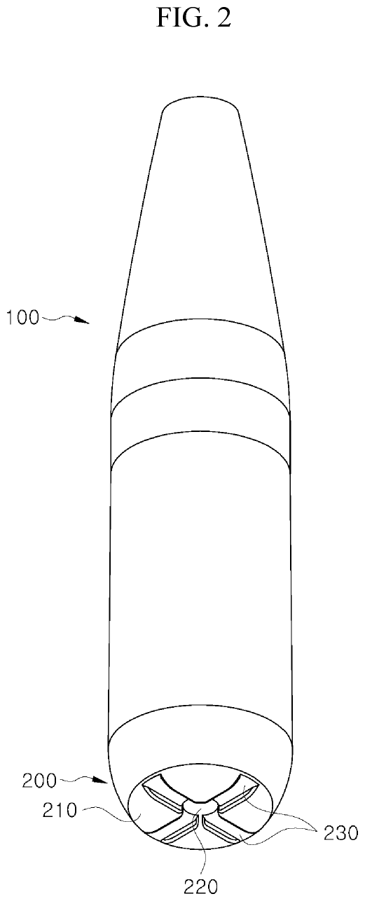

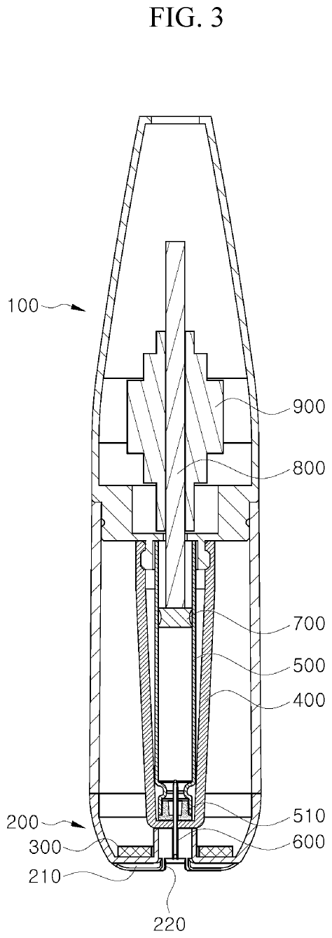

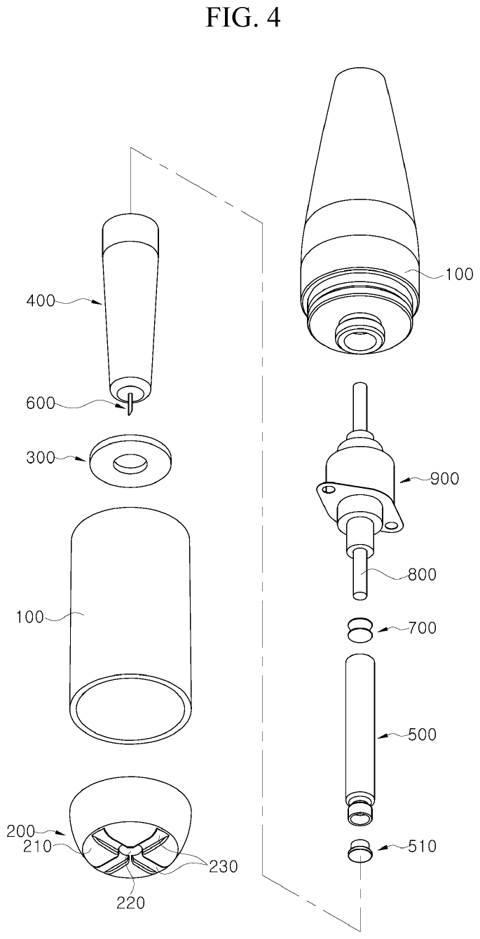

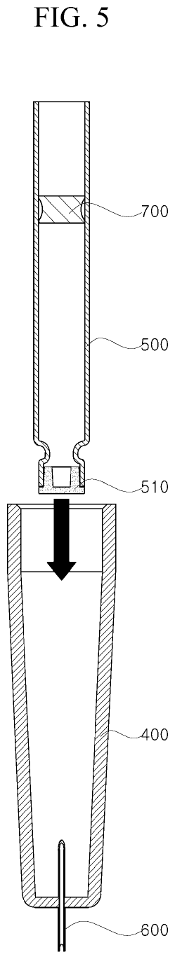

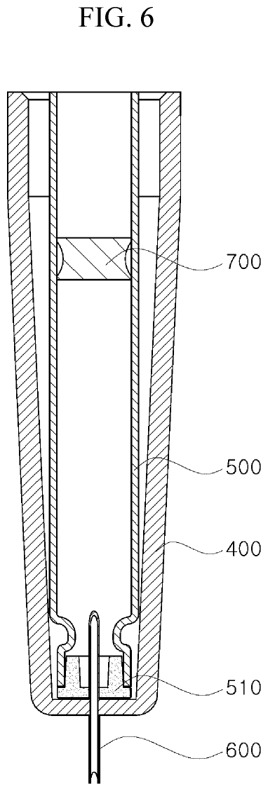

FIG. 1 is a perspective view illustrating a state in which a skin care device for both ultrasound delivery and drug application according to an embodiment of the present disclosure is in contact with a skin, FIG. 2 is a perspective view illustrating the skin care device for both ultrasound delivery and drug application according to the embodiment of the present disclosure, FIG. 3 is a cross-sectional view illustrating the skin care device for both ultrasound delivery and drug application according to the embodiment of the present disclosure, FIG. 4 is an exploded perspective view illustrating the skin care device for both ultrasound delivery and drug application according to the embodiment of the present disclosure, and FIGS. 5 to 6 are cross-sectional views illustrating a process of loading a drug solution container of a skin care device for both ultrasound delivery and drug application onto a container loading unit according to another embodiment of the present disclosure.

As illustrated in FIGS. 1 to 4, the skin care device for both ultrasound delivery and drug application according to the embodiment of the present disclosure includes a handpiece 100, a cartridge 200, an output unit 300, a container loading unit 400, a drug solution container 500, and a needle part 600.

The handpiece 100 and the cartridge 200, which are basic bodies, may serve as a handle that a user grips.

The cartridge 200 may be detachably coupled to the handpiece 100, and the cartridge 200 may be provided with the output unit 300 that outputs electrical energy including at least one of ultrasound, laser, light, a low-frequency microwave, a mid-frequency microwave, and a high-frequency microwave. Thus, in a state in which the cartridge 200 is in contact with a target part of a skin 10, while the cartridge 200 moves along the target part of the skin 10, the electrical energy including at least one of the ultrasound, the laser, the light, the low-frequency microwave, the mid-frequency microwave, and the high-frequency microwave, which are output from the output unit 300, is focused to a target depth of the skin 10, and then a medical procedure may be performed. However, it is most preferable that the output unit 300 outputs the electrical energy including the ultrasound.

In the embodiment, a plug (not illustrated) to which a power cable is connected may be provided at a rear end of the handpiece 100, and the tip of the handpiece 100 may be covered by a bottom cap. A connector insertion part to which a connector which will be described below is detachably coupled may be formed in the bottom cap of the handpiece 100.

In the embodiment, the handpiece 100 may be provided with a circuit board (not illustrated) that supplies power to the output unit 300 and controls the output unit 300, which will be described below, and a driving unit 900, which will be described below.

In the embodiment, the handpiece 100 may be provided with a stimulation output on/off button that switches the output unit 300, which will be described below, between an operating state and a stopping state in conjunction with the circuit board by pushing and a drug discharge on/off button that switches the driving unit 900, which will be described below, between an operating state and a stopping state in conjunction with the circuit board by pushing. As will be described below, when the output unit 300 is operated, the electrical energy including at least one of the ultrasound, the laser, the light, the low-frequency microwave, the mid-frequency microwave, and the high-frequency microwave may be output from the output unit 300 toward the target part of the skin 10, and when the driving unit 900 is operated, a drug solution stored in the drug solution container 500 may be discharged to the target part of the skin 10.

In the embodiment, the handpiece 100 may be divided into a first handpiece 100 body coupled to the cartridge 200, which will be described below, and a second handpiece 100 body in which the driving unit 900 which will be described below is accommodated, and the first handpiece 100 body and the second handpiece 100 body may be screw-coupled to each other. Further, the container loading unit 400 may be inserted into the first handpiece 100.

The cartridge 200, which is a type of housing that accommodates the output unit 300, may have a head part 210 in contact with the target part of the skin 10 Here, the cartridge 200 may have a cylindrical shape of which a diameter decreases in a direction approaching the head part 210.

The head part 210 may be provided on a bottom surface of the cartridge 200 and have a circular shape. A drug discharge portion 220 and a drug flow portion 230 may be formed in the head part 210.

The drug discharge portion 220 serves as a passage through which the drug solution delivered from the drug solution container 500, which will be described below, is discharged to the target part of the skin 10. For example, the drug discharge portion 220 may be formed through a central axis of the head part 210.

The drug flow portion 230 is recessed in the head part 210 in a direction perpendicular to the drug discharge portion 220 and serves to flow the drug discharged from the drug discharge portion 220 to the target part of the skin 10. In detail, in a state in which the head part 210 is in contact with the target part of the skin 10, the drug discharged from the drug discharge portion 220 to the target part of the skin 10 is moored at a specific point of the target part of the skin 10 in contact with the drug discharge portion 220 and then flows from the specific point of the target part of the skin 10 in contact with the drug discharge portion 220 to various points of the target part of the skin 10 in contact with the drug flow portion 230. That is, a drug delivery area of the target part of the skin 10 may flow through the drug flow portion 230.

In the embodiment, a plurality of drug flow portions 230 may be configured. Here, the plurality of drug flow portions 230 may be recessed radially from the drug discharge portion 220 in the head part 210.

The output unit 300 may be provided on the cartridge 200 and serve to output the electrical energy including at least one of the ultrasound, the laser, the light, the low-frequency microwave, the mid-frequency microwave, and the high-frequency microwave toward the target part of the skin 10. For example, the output unit 300 may have an annular shape and may be disposed around the drug discharge portion 220. In other words, a structure in which the drug discharge portion 220 passes along a central axis of the output unit 300 is provided. Thus, as the output unit 300 and the drug discharge portion 220 are provided in the cartridge 200 at the same height, the size of the cartridge 200 may be reduced, and thus a storage space of the cartridge 200 may be reduced.

The container loading unit 400 may be detachably coupled to the head part 210. The drug solution container 500 may be detachably loaded on the container loading unit 400. Thus, when it is required to replace the drug solution container 500 loaded on the container loading unit 400, after the drug solution container loaded on the container loading unit 400 is removed, a new drug solution container 500 may be loaded on the container loading unit 400.

In the embodiment, the container loading unit 400 may have a shape of which a rear end is open, a tip is closed, and a diameter decrease from the rear end to the tip. Thus, the drug solution container 500 may be easily inserted into the rear end of the container loading unit 400.

The drug solution container 500 may be loaded on the container loading unit 400 and store the drug solution. For example, the drug solution container 500 may be a vial. In detail, the vial may be a container in which the drug solution is injected to make a sterile state, and then a front end is sealed by a rubber stopper and a rear end is sealed by a rubber container packing 700. Here, the vial may be made of plastic or glass. Further, the container packing 700 may be provided to reciprocate in the vial, that is, the drug solution container 500. Further, the container packing 700 may reciprocate along the drug solution container 500 by a plunger 800, which will be described below.

The needle part 600 is provided in the container loading unit 400 to communicate with the container loading unit 400 and the drug discharge portion 220 and serves to apply the drug solution stored in the drug solution container 500 to the target part of the skin 10 through the drug discharge portion 220 by breaking a stopper of the drug solution container 500 loaded on the container loading unit 400. The needle part 600 may have a rear end protruding to the drug solution container 500 loaded on the container loading unit 400 and a tip protruding to the drug discharge portion 220, thereby playing the corresponding role. Thus, the needle part 600 may have the tip and the rear end that are sharp to perform the corresponding role. (see FIGS. 1 to 6)

The plunger 800 may push the container packing 700 in a direction approaching the needle part 600 to serve to discharge the drug solution stored in the drug solution container 500 to the drug discharge portion 220.

The driving unit 900 may be provided in the handpiece 100 and serve to move the plunger 800 in a direction approaching or away from the needle part 600. For example, a driving motor or actuator may be used in the driving unit 900.

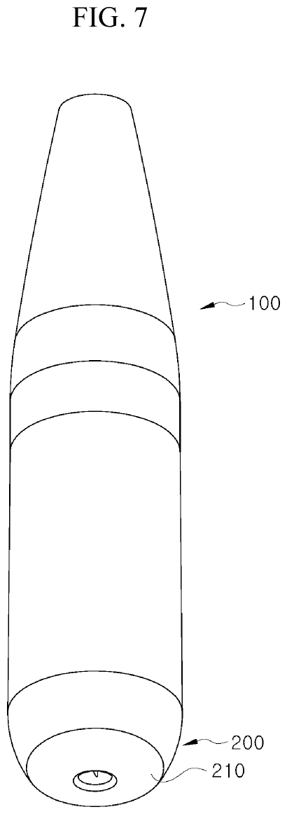

FIG. 7 is a cross-sectional view illustrating the skin care device for both ultrasound delivery and drug application according to another embodiment of the present disclosure.

As illustrated in FIG. 7, unlike the embodiment, the skin care device for both ultrasound delivery and drug application may exclude the drug flow portion of the head part 210 of the cartridge 200.

Hereinafter, an operation of the skin care device for both ultrasound delivery and drug application according to various embodiments of the present disclosure will be described. The following process may be performed by pushing the circuit board or an ultrasound output on/off button and the drug discharge on/off button provided in the circuit board.

First, after the drug solution container 500 is loaded on the container loading unit 400, the container loading unit 400 is mounted on the head part 210. In this case, the needle part 600 breaks the stopper of the drug solution container 500 loaded on the container loading unit 400.

Next, the head part 210 of the cartridge 200 is in contact with the target part of the skin 10.

Next, the output unit 300 and the driving unit 900 operate together. As a result, the electrical energy including at least one of the ultrasound, the laser, the light, the low-frequency microwave, the mid-frequency microwave, and the high-frequency microwave is output from the output unit 300 toward the target part of the skin 10. At the same time, the plunger 800 moves by the driving unit 900 in a direction approaching the needle part 600. Therefore, the plunger 800 pushes the container packing 700 in the direction approaching the needle part 600 to discharge the drug solution stored in the drug solution container 500 through the needle part 600 to the drug discharge portion 220, and thus the drug solution is applied to the target part of the skin 10.

According to the present disclosure, the skin care device for both ultrasound delivery and drug application according to the embodiment of the present disclosure may deliver the electrical energy and apply the drug solution to the skin 10 at the same time.

Hereinabove, embodiments of the present disclosure have been described with reference to the to the accompanying drawings. However, those skilled in the art to which the present disclosure pertains can understand that the present disclosure can be implemented in other specific forms without changing the technical spirit or essential features thereof. Therefore, it should be understood that the embodiments described above are illustrative but not limiting in all aspects.

What is claimed is:

1. A skin care device for both ultrasound delivery and drug application, the skin care device comprising:
a cartridge including a head part in contact with a target part of a skin and a drug discharge portion formed in the head part;
an output unit provided in the head part and configured to output electrical energy including at least one of ultrasound, laser, light, a low-frequency microwave, a mid-frequency microwave, and a high-frequency microwave toward the target part;
a handpiece detachably coupled to the cartridge;
a container loading unit embedded in the handpiece and detachably coupled to the head part;
a drug solution container which is loaded on the container loading unit and in which a drug solution is stored; and
a needle part provided in the container loading unit to communicate with the container loading unit and the drug discharge portion and configured to break a stopper of the drug solution container loaded on the container loading unit to apply the drug solution stored in the drug solution container to the target part,
wherein the cartridge includes a plurality of drug flow portions which are recessed radially from the drug discharge portion in the head part and through which the drug discharged from the drug discharge portion to the target part flows radially, and
the container loading unit has a shape of which a rear end is open, a tip is closed, and a diameter decreases from the rear end to the tip.

2. The skin care device of claim 1, further comprising:
a container packing provided in the drug solution container to reciprocate; and
a plunger configured to push the container packing in a direction approaching the needle part to discharge the drug solution stored in the drug solution container to the drug discharge portion.

3. The skin care device of claim 2, further comprising:
a driving unit provided in the handpiece and configured to move the plunger in a direction approaching or away from the needle part.

4. The skin care device of claim 1, wherein the output unit has an annular shape and is disposed around the drug discharge portion.
